(19) United States
(12) Patent Application Publication
(10) Pub. No.: US 2004/0067255 A1
(43) Pub. Date: Apr. 8, 2004

(54) Method of administering split doses of a vascular targeting agent

(76) Inventors: David J. Chaplin, Watlington (GB); Sally Hill, Chesham (GB)

Correspondence Address: MINTZ, LEVIN, COHN, FERRIS,, GLOVSKY and POPEO, P.C., One Financial Center, Boston, MA, 02111, US

(21) Appl. No.: 10/265,820

(22) Filed: Oct. 7, 2002

(51) Int. Cl. A61K 31/075; A61K 9/22

(52) U.S. Cl. 424/468; 514/720

(57) ABSTRACT

The present invention is directed to the use of vascular targeting agents or pharmaceutically acceptable salts thereof for administration in divided doses to a warm-blooded animal, such as a human. Also disclosed is a medicament comprising two or more fraction of doses of a vascular targeting agent, or a pharmaceutically acceptable salt thereof, which together add up to a total daily dose, or administration in divided doses for use in a method of treating a human or warm-blooded animal. A kit comprising two or more fractions of doses of a vascular targeting agent or a pharmaceutically acceptable salt thereof, which together add up to a total daily dose, for administration in divided doses is also disclosed.

### FIELD OF THE INVENTION

The present invention provides a method of administering Vascular Targeting Agents (“VTAs”) to treat diseases associated with malignant neovascularization.

### BACKGROUND OF THE INVENTION

Cancer is a leading cause of death in the industrialized world and despite years of research many types of cancer lack an effective therapeutic treatment. This is especially true for cancers that are characterized by the presence of large, solid tumors since it is difficult to deliver an effective dose of a chemotherapeutic agent to the interior of a large tumor mass with a degree of selectivity. Moreover, due to the genetic instability of tumor cells, a tumor tissue can rapidly acquire resistance to standard therapeutic regimens.

In order to develop into a large solid tumor mass however, a tumor foci must first establish a network of blood vessels in order to obtain the nutrients and oxygen required for further growth. This tumor-induced vasculature has received enormous interest as a target for antineoplastic therapy because a relatively small number of blood vessels are critical for the survival and continued growth of a much larger group of cancer cells. The disruption in the function of a single tumor blood vessel can result in an avalanche of ischaemic tumor cell death and necrosis of thousands of cancer cells which depend on it for blood supply. In addition, the accessibility of the tumor vasculature by blood-borne anticancer agents and the relatively stable genome of normal, host vascular tissue can alleviate some of the problems associated with conventional, anti-tumor based therapies.

Much of the research in anti-vascular cancer therapy has focused on understanding the process of new blood vessel formation, known as angiogenesis, and identifying anti-angiogenic agents which inhibit the formation of new blood vessels. Angiogenesis is characterized by the proliferation of tumor endothelial cells and generation of new vasculature to support the growth of a tumor. This growth is stimulated by certain growth factors produced by the tumor itself. One of these growth factors, Vascular Endothelial Growth Factor (“VEGF”), is relatively specific towards endothelial cells, by virtue of the restricted and up-regulated expression of its cognate receptor. Various anti-angiogenic strategies have been developed to inhibit this signaling process at one or more steps in the biochemical pathway in order to prevent the growth and establishment of the tumor vasculature. However, anti-angiogenic therapies act slowly and must be chronically administered over a period of months to years in order to produce a desired effect.

Vascular Targeting Agents (“VTAs”), also known as Vascular Damaging Agents, are a novel class of antineoplastic drugs which attack solid tumors by selectively targeting and destroying the existing neovasculature or vasculature newly formed by angiogenesis. The cytotoxic mechanism of VTA action is quite divorced from that of anti-angiogenic agents. A single dose of VTA can cause a rapid and selective shutdown of the tumor neovasculature within a period of minutes to hours, leading eventually to tumor necrosis by induction of hypoxia and nutrient depletion. Other agents have been known to disrupt tumor vasculature but differ in that they also manifest substantial normal tissue toxicity at their maximum tolerated dose. In contrast, genuine VTAs, such as the combretastatins, retain their vascular shutdown activity at a fraction of their maximum tolerated dose.

Combretastatin A-4 Disodium Phosphate Prodrug (“CA4DP”) having the following structure:

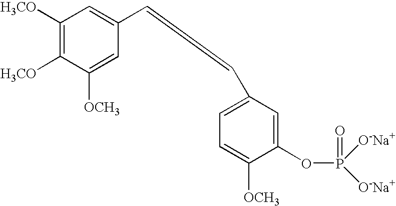

is the lead drug of a group of VTAs currently in clinical trials as a VTA. This compound was initially isolated as Combretastatin A-4 (“CA-4”) from the stem wood of the African tree Combretum caffrum (Combretaceae). As described in U.S. Pat. No. 4,996,237, the entire disclosure of which is incorporated herein in entirety, CA-4 which has the structure:

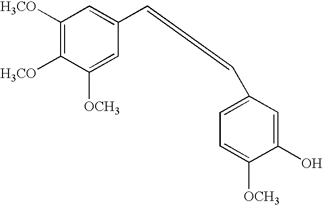

was synthesized and found to have tubulin binding activity. Moreover, CA4DP was found to be a potent inhibitor of microtubule assembly in tumor endothelium. However, due to the insolubility of CA-4 in human plasma, CA4DP was developed and found to have superior activity as a VTA (U.S. Pat. No. 5,561,122, the entire disclosure of which is incorporated by reference). When administered to the bloodstream of a patient, the CA4DP is cleaved to the active, tubulin-binding CA-4 by endogenous nonspecific phosphatases. It is thought that CA-4 selectively destabilizes the microtubule cytoskeleton of tumor endothelial cells, causing a profound alteration in the shape of the cell which ultimately leads to occlusion of the tumor blood vessel and shutdown of blood flow to the tumor (Kanthou and Tozer, Blood, 2002, 99(6): 2060-2069).

While in vivo studies have confirmed that vascular damaging effects of VTAs on tumor tissue far exceed the effects on normal tissues, only in a few cases has a tumor regression or complete tumor response been observed when these agents are used alone as a monotherapy. The lack of traditional tumor response has been attributed to the rapid recolonization of the necrotic tumor core by a viable rim of well-oxygenated tumor cells which survive the effects of vascular targeting (Chaplin, et al., Anticancer Research, 1999, 19(1A):189-195). While this viable rim is resistant to VTA therapy, it remains highly susceptible to conventional radiation, chemotherapy and antibody-based therapeutics, and many studies have demonstrated effective tumor regression when VTAs are used in combination with one of these therapies (Li and Rojiani, Int. J. Radiat. Oncol. Biol. Phys., 1998, 42(4): 899-903; Grosios et al., Anticancer Research, 2000, 20(1 A): 229-233; Pedley et al., Cancer Research, 2001, 61(12): 4716-4722; WO 02/056692).

Despite the effectiveness when used in combination with VTA therapy, conventional therapies must be administered in repeat daily doses following initial VTA administration in order to achieve prolonged tumor regression. Most conventional therapies are highly cytotoxic, and the patient most cope with prolonged side effects (emesis, hair loss, myelosuppression, etc.) due chronic administration. VTA therapies lack many of these toxic effects. There is therefore an urgent need in the art for an effective therapy in which the VTA is administered as a single agent.

### SUMMARY OF THE INVENTION

The present invention relates to a method of administering a vascular targeting agent in divided (or split) doses to treat diseases associated with malignant neovascularization.

In a first embodiment, the present invention is directed to a method for producing an anti-tumor effect in a warm-blooded animal such as a human, by administration of divided doses of an effective amount of a vascular targeting agent or a pharmaceutically acceptable salt thereof. The present invention particularly relates to such a method wherein the vascular targeting agent is a combretastatin or analog thereof.

In another embodiment, the present invention is directed to a medicament comprising two or more fraction of doses of a vascular targeting agent or a pharmaceutically acceptable salt thereof which together add up to a total daily dose, or administration in divided doses for use in a method of treating a human or warm-blooded animal.

The present invention also relates to a kit comprising two or more fractions of a vascular targeting agent or a pharmiaceutically acceptable salt thereof, which together add up to a total daily dose, for administration in divided doses.

The details of one or more embodiments of the invention are set forth in the accompanying description below. Although any methods and materials similar or equivalent to those described herein can be used in the practice or testing of the present invention, the preferred methods and materials are now described. Other features, objects, and advantages of the invention will be apparent from the description. In the specification and the appended claims, the singular forms also include the plural unless the context clearly dictates otherwise. Unless defined otherwise, all technical and scientific terms used herein have the same meaning as commonly understood by one of ordinary skill in the art to which this invention belongs. All patents and publications cited in this specification are incorporated herein by reference

### DETAILED DESCRIPTION OF THE FIGURES

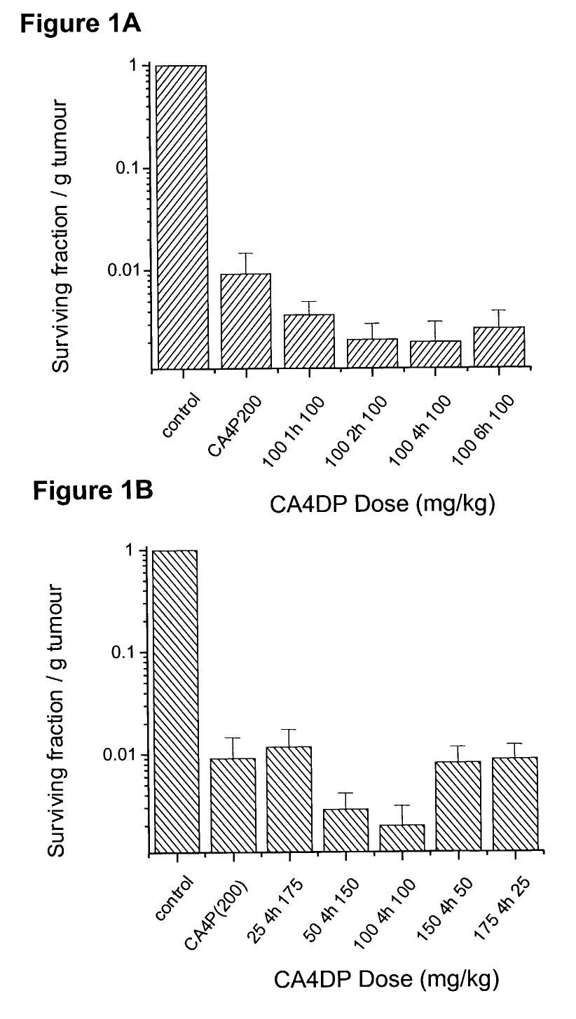

FIG. 1A graphically illustrates that the surviving fraction of tumor cells by administration of two equal doses of 100 mg/kg CA4DP, separated by 1-6 hours, produced significantly more cell killing than the administration of a single 200 mg/kg dose of CA4DP;

FIG. 1B graphically illustrates that administration of CA4DP in unequal doses, separated by 4 hours was less effective than equal doses of treatment;

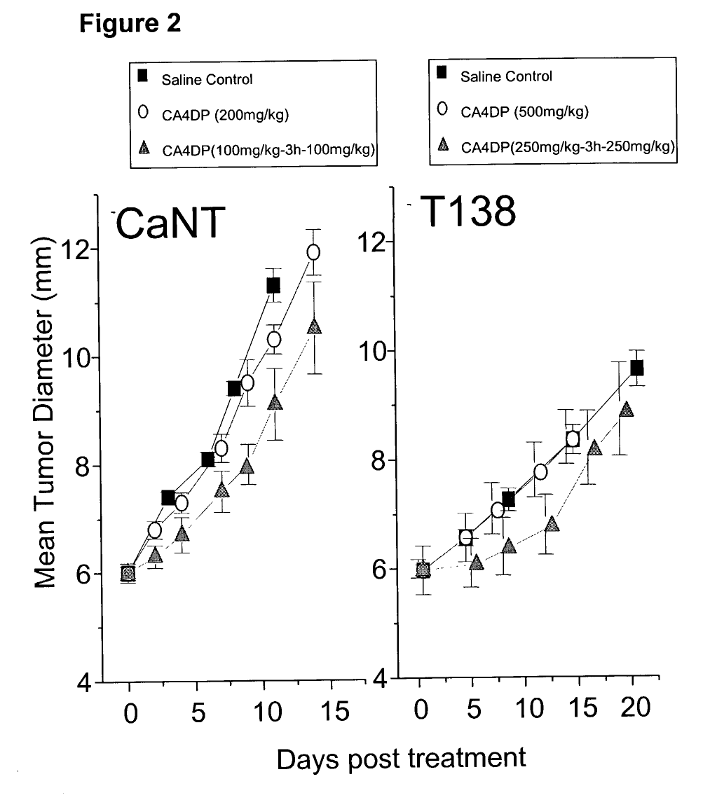

FIG. 2 graphically illustrates the results of split dose therapy of CA4DP on tumor growth delay;

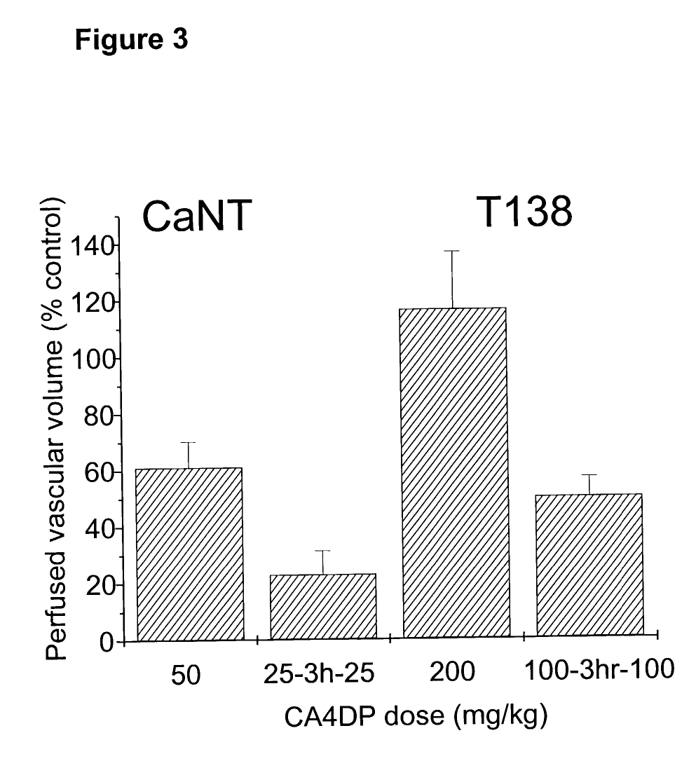

FIG. 3 graphically illustrates that the administration of two equal split doses of CA4DP were significantly more effective than a single dose at reducing the fraction of perfused vascular volume; and

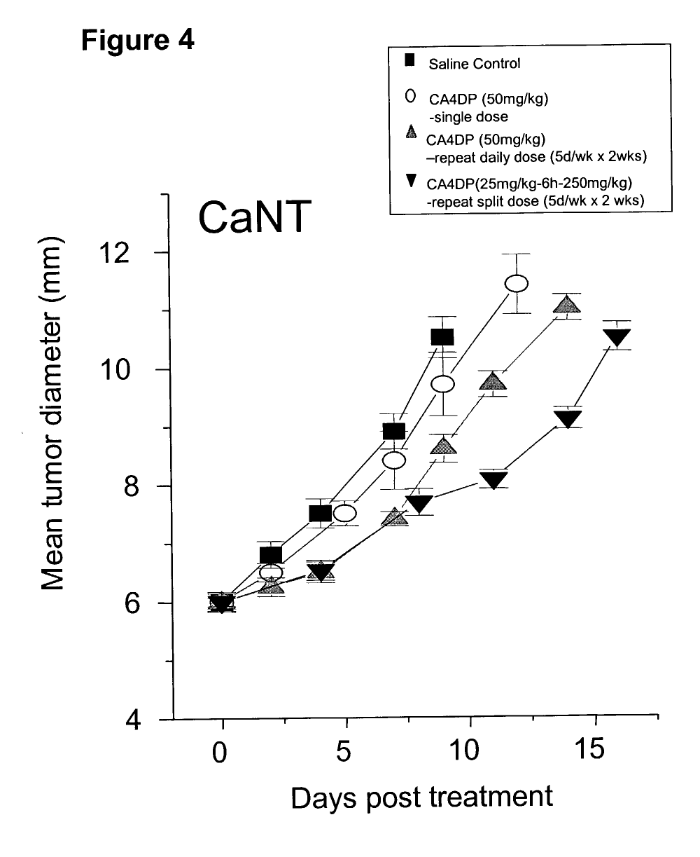

FIG. 4 illustrates the administration of a large single dose of CA4DP (50 mg/kg) produced only minimal effects on tumor growth relative to control.

### DETAILED DESCRIPTION OF THE INVENTION

The present invention provides, in a first aspect, a method of administering in an effective amount of VTA to a patient suffering from neovascularization such that the anti-tumor effect of single agent treatment is enhanced. The inventors have made the unexpected and surprising discovery that it is possible to achieve a prolonged anti-tumor effect using a VTA as a single agent if the drug is administered in a multiple fractions of total dose (i.e. split or divided doses) instead of a single bolus of the total daily dose. In another aspect of the invention, the inventors have discovered that if the split-dose is administered repeatedly for several consecutive days, the anti-tumor effect is enhanced to an even greater degree. This prolonged anti-tumor effect of may include, but is not limited to, tumor growth delay, reduced vascular perfusion of the tumor, tumor regression, tumor shrinkage, increased time to regrowth of tumor on cessation of treatment, and slowing of disease progression. While not wishing to be limited to a particular mechanism, this anti-tumor effect may be attributed to the initial blood flow shutdown and subsequent necrosis and/or additional anti-proliferative effects on the tumor and endothelial cells, which retard the revascularization and repopulation of the tumor core by the viable rim of cells that are unaffected by blood-flow shutdown.

According to yet another aspect of the invention, there is provided a medicament comprising two or more fractions of doses of VTA, which together add up to the total daily allowable dose. In yet another aspect of the invention, there is provided a kit comprising two or more fractions of doses of VTA, which together add up to the total daily dose, for administration in a divided dose schedule. The kit may optionally consist of a container means for containing the dose fractions.

In accordance with the present invention, the preferred VTA is CA4DP, a disodium salt of the phosphate prodrug of CA-4. The invention is not limited in this respect, however, and other phosphate prodrug salts of CA-4 such as those disclosed in WO 02/22626 and WO 99/35150 may work as well or better than CA4DP.

The invention also contemplates the use of other Combretastatins that have been isolated, structurally elucidated, and synthesized. U.S. Pat. Nos. 5,409,953, 5,569,786, and 4,490,726 describe the isolation and synthesis of Combretastatins designated as A-1, A-2, A-3, B-1, B-2, B-3, B-4, D-1, and D-2. Some of these compounds have been modified as phosphate prodrugs as disclosed in WO 01/81355. The disclosures of these references are incorporated herein in their entirety.

Furthermore, the present invention contemplates the use of synthetic analogs of the Combretastatins as described in Bioorg. Med. Chem. Lett. 11(2001) 871-874, 30733076, J. Med. Chem. (2002), 45:1697-1711, WO 01/12579, WO 00/35865, WO 00/48590, WO 01/12579, U.S. Pat. No. 5,430,062, U.S. Pat. No. 5,525,632, U.S. Pat. No. 5,674,906, and U.S. Pat. No. 5,731,353, the entire disclosures of which are incorporated herein by reference.

Other tubulin binding agents which may be administered as VTAs include the following agents or their prodrugs: 2,3-disubstituted Benzo[b]thiophenes (U.S. Pat. Nos. 5,886,025; 6,162,930, and 6,350,777), 2,3-disubstituted benzo[b]furans (WO 98/39323), 2-3-disubstituted indoles (WO 01/19794), disubstituted dihydronaphthalenes (WO01/68654), Colchicine analogs (WO 99/02166, WO 00/40529, WO 02/04434, WO 02/08213), Chalcone analogs (WO 02/47604) the entire disclosures are incorporated by reference herein. Finally, additional non-cytotoxic prodrugs of tubulin binding agents, which are converted to a substantially cytotoxic drug by action of an endothelial enzyme are disclosed in WO 00/48606, which is incorporated by reference here.

In preferred embodiment of the invention, the dose fraction is divided into at least 10 equal or nonequal fractions of total daily dose. More preferably, the agent is divided into two equal fractions of total daily dose.

Split or divided doses as used herein means that the total dose to be administered to a warm-blooded animal, such as a human, in any single day period (for example one 24 hour period from midnight to midnight) is divided up into two or more fractions of the total dose and these fractions are administered with a time interval between each fraction of greater than 0 minutes to about 24 hours, preferably between approximately 1 hour to approximately 6 hours, more preferably approximately 2 hours to approximately 4 hours. The fractions of total dose may be about equal or unequal. If more than two doses are administered, the time intervals between each dose may be about equal or unequal.

In yet another preferred embodiment of the invention, VTA is administered in a multiple unit dosage forms to a total daily dose of between approximately 2 and approximately 1000 mg per kg. More preferably, the total daily dose administered is in the range of approximately 10-500 mg/kg. Most preferably, the total daily dose administered is between approximately 30-60 mg/kg.

The total daily dose which is required for the therapeutic or prophylactic treatment of a particular disease state will necessarily be varied depending on the host treated, the route of administration and the severity of the illness being treated. Accordingly, the optimum dosage maybe determined by the practitioner who is treating any particular patient.

The VTA is advantageous in a form suitable for parenteral injection (including intravenous, subcutaneous, intramuscular, intravascular or infusion) for example as a sterile solution, suspension or emulsion, but may also be in a form suitable for oral administration, for example as a tablet or capsule, for nasal administration or administration by inhalation, for example as a powder or solution, for topical administration for example as an ointment or cream, for rectal administration for example as a suppository or the route of administration may be by direct injection into the tumor or by regional or local delivery. In other embodiments of the present invention, the VTA may be delivered endoscopically, intratracheally, intralesionally, percutaneously, intravenously, subcutaneously, intraperitoneally or intratumorally. The VTA may be in the form of a pharmaceutical composition wherein the VTA or a pharmaceutically acceptable salt thereof is in association with a pharmaceutically acceptable excipient or carrier. In general the compositions described herein may be prepared in a conventional manner using conventional excipients. The compositions of the present invention are advantageously presented in unit dosage form.

The invention will now be illustrated by the following non-limiting examples and with reference to the accompanying figures. All examples outline experiments performed in animal models and in no way be construed as limiting scope of the invention to these animals. For the avoidance of doubt the term “patient” in the description refers to any warm-blooded animal.

One or both of following animal tumor models were used in Examples 1-4:

1) Transplanted tumor model. A murine adenocarcinoma NT (“CaNT”) tumor was transplanted from a syngeneic donor and implanted subcutaneously onto the rear dorsum of 10-16 week old female CBA/Gy f TO mice, by injecting 0.05 ml of a crude cell suspension prepared by mechanical dissociation of the excised. Tumors were selected for treatment with the VTA CA4DP when the geometric mean diameter (“GMD”) reached 5-6.5 mm (150-300 mg), approximately 3-4 weeks after implantation.

2) Spontaneous T138 tumor model. The T138 tumor-bearing mice (“T138”) spontaneously developed mammary tumors between 5 and 18 months of age due to genetic predisposition. Tumors displayed of growth rates (4.5 to 20 days volume doubling time at approx. 6 mm GMD; mean=10.2±0.9 days)

In each of the following Examples 1-4, CA4DP (OXiGENE Inc, Watertown, Mass., USA) was dissolved in 0.9% saline at appropriate concentrations to allow each dose to be injected i.p. in 0.1 ml per 10 g body weight. 0.9% saline was used for control treatments.

### EXAMPLE 1

### Effect of Split VTA Dosing on Tumor Cell Survival

Methods and Materials

To compare the anti-tumor effect of single and divided VTA dose scheduling, the survival of tumor cells isolated from a treated CaNT tumor-bearing animals was assessed by an in vitro clonogenic (colony-forming) assay, as described previously (Chaplin et al., Anticancer Research, 1999, 19: 189-196). Tumors were excised 18-24h after CA4P injection. Two tumors were combined for each data point, weighed, minced with scissors and then disaggregated for 1 hour at 37° C. in an enzyme cocktail of 1 mg/ml pronase, 0.5 mg/ml DNase and 0.5 mg/ml collagenase. Following digestion, samples were passed through a 25G needle and a 35 μm filter to obtain a single cell suspension. Haemocytometer counts of trypan-blue excluding cells were made and known numbers of viable cells added to a feeder layer of heavily irradiated V79-379A Chinese hamster cells. After 7-10 days incubation, colonies were fixed, stained with methylene blue and counted. The data were calculated as surviving fraction per gram of tumor, which is a product of relative surviving fraction and relative cell yield per gram of tumor. Each experimental group was repeated at least 3 times, so that at least 6 tumors contribute to each data point.

Results

The results of the cell survival are depicted in FIGS. 1A and 1B. As evidenced by the surviving fraction of tumor cells, the administration of two equal doses of 100 mg/kg CA4DP, separated by 1-6 hours, produced significantly more cell killing than the administration of a single 200 mg/kg dose of CA4DP (FIG. 1A). The effect was most pronounced when the time interval between doses was 2 to 4 hours (p<0.05). Administration of CA4DP in unequal doses, separated by 4 hours, was less effective than equal doses of treatment (FIG. 1B).

### EXAMPLE 2

### Effect of Split VTA Dosing on Tumor Growth Delay

Material and Methods

The anti-tumor effect of split dosing therapy was also assessed by an analysis of tumor growth delay in both CaNT and T138 tumor-bearing mice. For experiments involving the CaNT, dose groups comprised 5 to 6 animals, whereas for the T138, 10 to 15 animals were used. Following initial drug treatment, tumor growth was determined by measuring the diameter of each tumor in 3 orthogonal orientations two or three days each week.

Results

The results of split dose therapy on tumor growth delay are illustrated in FIG. 2. As in Example 1, equal split dose therapy resulted in an enhanced tumor response over a single dose of CA4DP. This was apparent in the CaNT mice, where a single 200 mg/kg dose of CA4DP was compared with two equal doses of 100 mg/kg separated by a time interval of 3 hours. A single dose of CA4DP dose did not delay growth relative to control, while split dose therapy delayed tumor growth for a period of approximately 3 days. In the more resistant spontaneous T138 tumors, two equal doses of 250 mg/kg CA4DP, separated by a time interval of 3 hours, delayed tumor growth for approximately 4 days, while a single dose of 500 mg/kg had no effect on tumor growth.

### EXAMPLE 3

### Effect of Split VTA Dosing on the Vascular Perfusion of Tumors

The effect of VTA dose splitting on the tumor vascular response was also investigated in both CaNT and T138 tumor types, by measuring the functional vascular volume at 24 hours following treatment with single or split doses of CA4DP. Functional vascular volume is defined as the volume of tumor tissue perfused by tumor blood vessels, and is measured using the fluorescent DNA-binding dye Hoechst 33342 (Sigma-Aldrich Company Ltd., Dorset, UK) (Smith et al, British Journal of Cancer, 1988, 57: 247-253). The dye was dissolved in 0.9% saline at 6.25 mg/ml and injected i.v. at a dose of 10 mg/kg at 24 hours post-treatment. Tumors were excised and frozen 1 minute later. Sections were cut at three levels and observed under UV illumination. Functional vessels were identified by the fluorescent outline of perivascular tissue and vascular volumes were determined using a random point scoring system based on that described by Chalkley (Chalkley, J. Natl. Cancer Inst., 1943, 4: 47-53). At least 100 fields were scored at each of the three tumor levels and the results for treated tumors were expressed as a percentage of control values.

As outlined in FIG. 3, administration of two equal split doses of CA4DP were significantly more effective than a single dose at reducing the fraction of perfused vascular volume. This enhanced effect was observed in both the CaNT and T138 tumor models. In the CaNT tumor model, administration two 25 mg/kg doses, separated by a 3 hour time interval, resulted in a 80% reduction in perfused vascular volume, whereas a single 50 mg/kg dose was only 40% effective. In the more resistant T138 model, a single dose 200 mg/kg CA4DP was completely ineffective in reducing volume of perfused vascular volume. When administered in two split doses of 100 mg/kg, the amount of perfused vascular tissue was dramatically reduced to 50% of the control value.

### EXAMPLE 4

### Effect of Repeated Split VTA Dosing on Tumor Growth Delay

The effect of repeated split VTA dosing therapy was also assessed by an analysis of tumor growth delay in both CaNT tumor-bearing mice. As FIG. 4 demonstrates, administration of a large single dose of CA4DP (50 mg/kg) produced only minimal effects on tumor growth relative to control. Repeated administration of total daily dose (50 mg/kg) 5 days a week for 2 weeks, produced a significant delay in tumor growth of approximately 2 days. Tumor growth delay was further improved by repeat administration of split dosing (2 doses of 25 mg/kg per day separated by a 6 hour interval), 5 days a week for 2 weeks (growth delay of approximately 5 days).

### Other Embodiments

It is to be understood that while the invention has been described in conjunction with the detailed description thereof, the foregoing description is intended to illustrate and not limit the scope of the invention, which is defined by the scope of the appended claims.

It is also to be understood that the drawings are not necessarily drawn to scale, but that they are merely conceptual in nature.

What is claimed is:

1. A method for producing an anti-tumor effect in a warm-blooded animal, comprising administering in divided doses to said animal an effective amount of a vascular targeting agent or a pharmaceutically acceptable salt thereof.

2. The method according to claim 1, wherein a total daily dose of the vascular targeting agent is divided into two or more parts which are about equal or unequal with a time interval between doses of greater than or equal to approximately one hour and less than or equal to approximately six hours.

3. The method according to claim 1, wherein the total daily dose of the vascular targeting agent is divided into two or more parts which are about equal or unequal with a time interval between doses of greater than or equal to approximately two hours and less than or equal to approximately four hours.

4. The method according to claim 1, wherein the total daily dose of the vascular targeting agent is less than or equal to 500 mg/kg.

5. The method according to claim 1, wherein the total daily dose of the vascular targeting agent is in the range between approximately 30 mg/kg to approximately 60 mg/kg.

6. The method according to claim 1, wherein the divided doses are administered in a repeated dose schedule.

7. The method according to claim 1, wherein the vascular targeting agent is a combretastatin or an analog thereof.

8. The method according to claim 1, wherein the warm-blooded animal is a human.

9. A method according to claim 7, wherein the total dose of the combretastatin is divided into two or more parts which are about equal or unequal with a time interval between doses of greater than or equal to approximately one hour and less than or equal to approximately six hours.

10. The method according to claim 7, wherein the total dose of combretastatin is divided into two or more parts which are about equal or unequal with a time interval between doses of greater than or equal to approximately two hours and less than or equal to approximately four hours.

11. The method according to claim 7, wherein the total daily dose of combretastatin is less than or equal to 500 mg/kg.

12. The method according to claim 7, wherein the total daily dose of the vascular targeting agent is in the range between approximately 30 mg/kg to approximately 60 mg/kg.

13. The method according to claim 7, wherein the divided doses are administered in a repeated dose schedule.

14. The method according to claim 6, wherein the combretastatin is a phosphate prodrug salt of combretastatin A-4.

15. The method according to claim 14, wherein the total dose of the phosphate prodrug salt is divided into two or more parts which are about equal or unequal with a time interval between doses of greater than or equal to approximately one hour and less than or equal to approximately six hours.

16. The method according to claim 14, wherein the total dose of phosphate prodrug salt is divided into two or more parts which are about equal or unequal with a time interval between doses of greater than or equal to approximately two hours and less than or equal to approximately four hours.

17. The method according to claim 14, wherein the total daily dose of combretastatin is less than or equal to 500 mg/kg.

18. The method according to claim 14, wherein the total daily dose of the vascular targeting agent is in the range between approximately 30 mg/kg to approximately 60 mg/kg.

19. The method according to claim 14, wherein the divided doses are administered in a repeated dose schedule.

20. A medicament comprising two or more fraction of doses of a vascular targeting agent or a pharmaceutically acceptable salt thereof, which together add up to a total daily dose, or administration in divided doses for use in a method of treating a human or warm-blooded animal by therapy.

21. The medicament according to claim 20, wherein the vascular targeting agent is a combretastatin or an analog thereof.

22. The medicament according to claim 21, wherein the combretastatin is a phosphate prodrug salt of combtetastatin A-4.

23. A kit comprising two or more fractions of doses of a vascular targeting agent or a pharmaceutically acceptable salt thereof, which together add up to a total daily dose, for administration in divided doses.

24. The kit according to claim 23, wherein the vascular targeting agent is a combretastatin or analog thereof.

25. The kit according to claim 23, wherein the combretastatin is a phosphate prodrug salt of combretastatin A-4.

26. A method for producing an anti-tumor effect in a warm-blooded animal, comprising administering in divided doses to said animal an effective amount of combretastatin or analog thereof, or a pharmaceutically acceptable salt thereof.

27. The method according to claim 26, wherein a total daily dose of the vascular targeting agent is divided into two or more parts which are about equal or unequal with a time interval between doses of greater than or equal to approximately one hour and less than or equal to approximately six hours.

28. The method according to claim 26, wherein the total daily dose of the vascular targeting agent is divided into two or more parts which are about equal or unequal with a time interval between doses of greater than or equal to approximately two hours and less than or equal to approximately four hours.

29. The method according to claim 26, wherein the total daily dose of the vascular targeting agent is less than or equal to 500 mg/kg.

30. The method according to claim 26, wherein the total daily dose of the vascular targeting agent is in the range between approximately 30 mg/kg to approximately 60 mg/kg.

31. The method according to claim 26, wherein the divided doses are administered in a repeated dose schedule.

32. The method according to claim 26, wherein the vascular targeting agent is a combretastatin or an analog thereof.

33. The method according to claim 26, wherein the warm-blooded animal is a human.

34. The method according to claim 26, wherein the combretastatin is a phosphate prodrug salt of combretastatin A-4.
